(19) United States
(12) Patent Application Publication
(10) Pub. No.: US 2016/0198938 A1
(43) Pub. Date: Jul. 14, 2016

(54) VIDEO CAMERA AND SPECULUM COMBINATION ASSEMBLY

(71) Applicant: Elvire Lizaire, Henderson, NC (US)

(72) Inventor: Elvire Lizaire, Henderson, NC (US)

(21) Appl. No.: 14/592,966

(22) Filed: Jan. 9, 2015

(51) Int. Cl. A61B 1/32; A61B 1/045; A61B 1/00; A61B 1/05; A61B 1/06

(52) U.S. Cl. CPC A61B 1/32; A61B 1/051; A61B 1/0684; A61B 1/00022; A61B 1/0676; A61B 1/045; A61B 1/00066; A61B 1/00029; A61B 1/00114; A61B 1/00124

(57) ABSTRACT

A video camera and speculum combination assembly includes a speculum that has a jaw portion and a handle portion. A housing is provided. A camera is coupled to the housing and the housing is coupled to the speculum. The camera may record video of an examination performed with the speculum. A plurality of light emitters is coupled to the housing. The light emitters illuminate the examination performed with the speculum. An actuator is mounted to the housing and is electrically coupled to the camera and the light emitters. An electronic memory is removably coupled to the housing. The electronic memory is in electrical communication with the camera such that electronic memory stores video recorded by the camera. A power supply is electrically coupled to the actuator.

### BACKGROUND OF THE DISCLOSURE

### Field of the Disclosure

The disclosure relates to speculum devices and more particularly pertains to a new speculum device for recording a video of an examination performed with the speculum.

### SUMMARY OF THE DISCLOSURE

An embodiment of the disclosure meets the needs presented above by generally comprising a speculum that has a jaw portion and a handle portion. A housing is provided and a camera is coupled to the housing. The housing is coupled to the speculum. The camera records video of an examination performed with the speculum. A plurality of light emitters is coupled to the housing. The light emitters illuminate the examination performed with the speculum. An actuator is mounted to the housing. The actuator is electrically coupled to the camera and the light emitters. An electronic memory is removably coupled to the housing and is in electrical communication with the camera such that electronic memory stores video recorded by the camera. A power supply is electrically coupled to the actuator.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a left side view of a video camera and speculum combination assembly according to an embodiment of the disclosure.

FIG. 2 is a perspective view of an embodiment of the disclosure.

FIG. 3 is a perspective view of a housing of an embodiment of the disclosure.

FIG. 4 is a bottom view of an embodiment of the disclosure.

FIG. 5 is a schematic view of an embodiment of the disclosure.

### DESCRIPTION OF THE PREFERRED EMBODIMENT

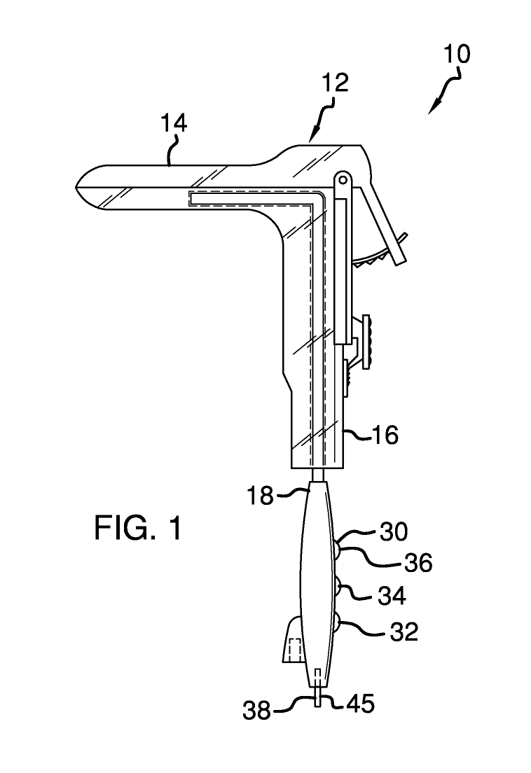

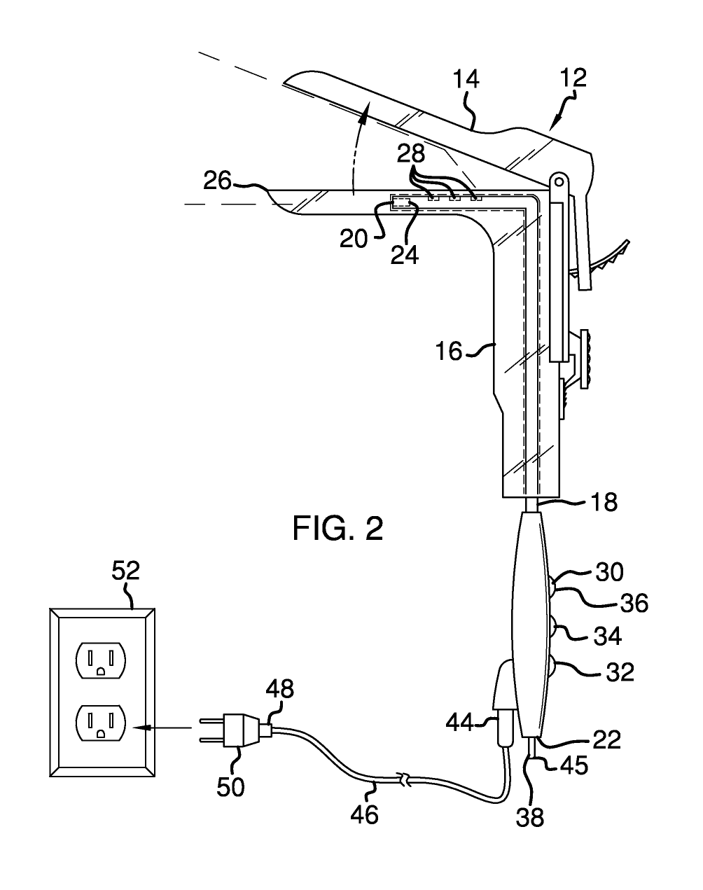

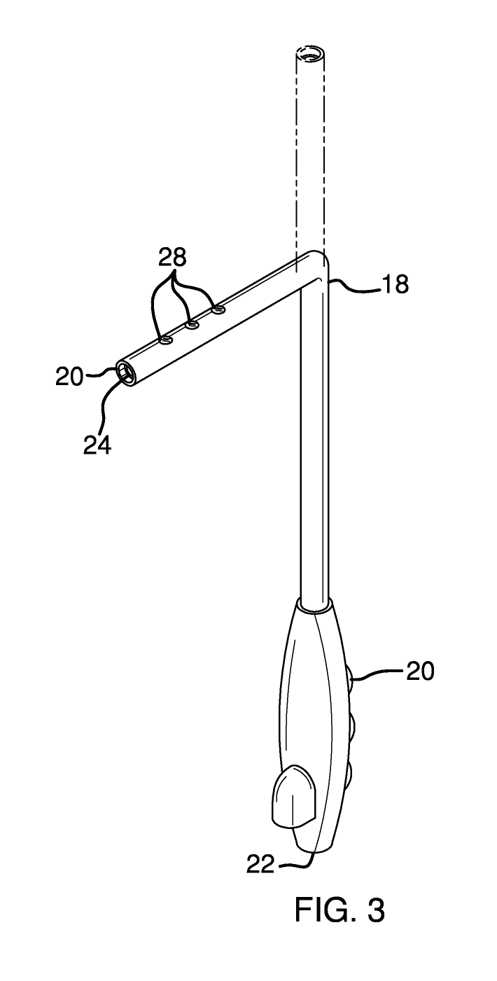

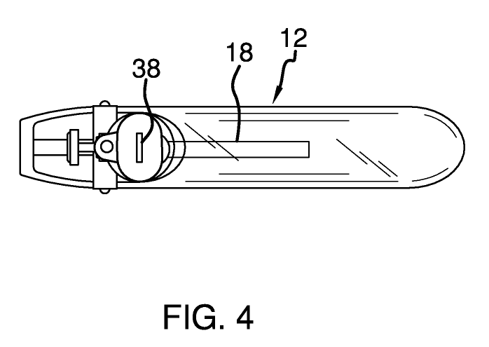

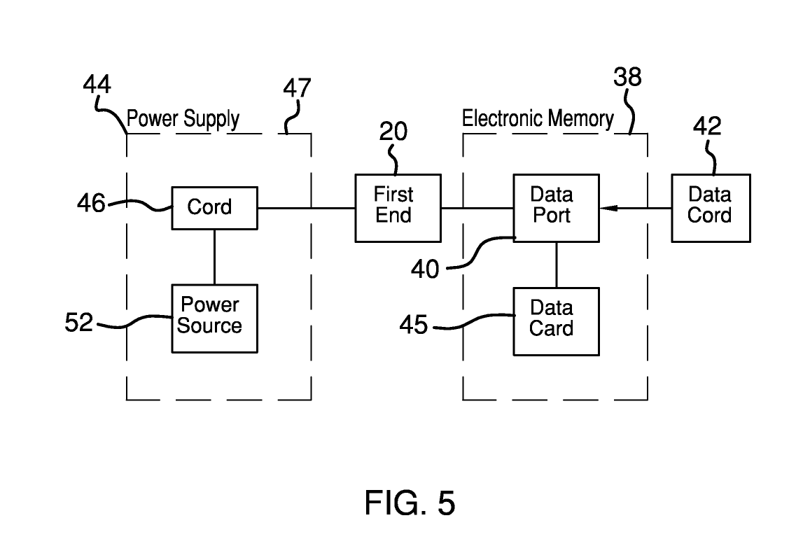

With reference now to the drawings, and in particular to FIGS. 1 through 5 thereof, a new speculum device embodying the principles and concepts of an embodiment of the disclosure and generally designated by the reference numeral 10 will be described.

As best illustrated in FIGS. 1 through 5, the video camera and speculum combination assembly 10 generally comprises a speculum 12 that has a jaw portion 14 and a handle portion 16. A housing 18 has a first end 20 and a second end 22. The housing 18 is elongated between the first end 20 and the second end 22. The housing 18 is comprised of a flexible material.

A camera 24 is coupled to the first end 20. The camera 24 may be a digital video camera of any conventional design. The housing 18 is positionable to extend through the handle portion 16 and the jaw portion 14. Moreover, the first end 20 is positioned proximate a free end 26 of the jaw portion 14. The camera 24 may record video of an examination performed with the speculum 12.

A plurality of light emitters 28 may be coupled to an outer wall 29 the housing 18. The light emitters 28 are distributed from the first end 20 toward the second end 22. The light emitters 28 illuminate the examination performed with the speculum 12. Each of the light emitters 28 may comprise an LED or the like. The light emitters 28 may be oriented perpendicular to the camera 24.

An actuator 30 is mounted to the housing 18 and is electrically coupled to the camera 24 and the light emitters 28. The actuator 30 may be one of a plurality of actuators 30 that includes an “on” actuator 32, a camera actuator 34 and a light actuator 36.

An electronic memory 38 is removably coupled to the housing 18. The electronic memory 38 is in electrical communication with the camera 24 such that electronic memory 38 stores video recorded by the camera 24. The electronic memory may comprise a data card 45. The electronic memory 38 may alternatively be permanently mounted in the housing 18. The housing 18 may then have a data port 40 electrically coupled thereto and electrically coupled to the electronic memory 38. The data port 40 may receive a data cord 42 to download the video data from the electronic memory 38.

A power supply 44 is electrically coupled to the camera 24. The power supply 44 may comprise a cord 46 extending away from the housing 18. The power supply may additionally comprise a battery 47. The cord 46 has a distal end 48 with respect to the housing 18. The distal end 48 has a plug 50 electrically coupled thereto. The plug 50 is configured to be electrically coupled to a power source 52. The power source 50 may be an electrical outlet or the like.

In use, the speculum 12 is employed in the convention of gynecological examination. The camera 24 records video of the examination and the video is downloaded from the electronic memory 38. The video is then viewable for diagnostic and training purposes.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A video camera and speculum combination assembly comprising:
a speculum having a jaw portion and a handle portion;
a housing;
a camera coupled to said housing, said housing being coupled to said speculum, said camera being configured record video of an examination performed with said speculum;
a plurality of light emitters coupled to said housing, said light emitters being configured to illuminate the examination performed with said speculum;
an actuator mounted to said housing, said actuator being electrically coupled to said camera and said light emitters;
an electronic memory removably coupled to said housing, said electronic memory being in electrical communication with said camera such that electronic memory stores video recorded by said camera; and
a power supply electrically coupled to said actuator.

2. The assembly according to claim 1, further comprising said housing having a first end and a second end, said housing being elongated between said first end and said second end, said housing being comprised of a flexible material.

3. The assembly according to claim 2, wherein said housing is positionable to extend through said handle portion and said jaw portion such that said first end is positioned proximate a free end of said jaw portion.

4. The assembly according to claim 2, wherein said light emitters are positioned adjacent to said first end.

5. The assembly according to claim 1, wherein said power supply comprises a cord extending away from said camera, said cord having a distal end with respect to said camera, said distal end having a plug electrically coupled thereto, said plug being configured to be electrically coupled to a power source.

6. A video camera and speculum combination assembly comprising:
a speculum having a jaw portion and a handle portion;
a housing having a first end and a second end, said housing being elongated between said first end and said second end, said housing being comprised of a flexible material;
a camera coupled to said first end, said housing being positionable to extend through said handle portion and said jaw portion such that said first end is positioned proximate a free end of said jaw portion, said camera being configured record video of an examination performed with said speculum;
a plurality of light emitters coupled to said housing, said light emitters being positioned adjacent to said first end, said light emitters being configured to illuminate the examination performed with said speculum;
an actuator mounted to said housing, said actuator being electrically coupled to said camera and said light emitters;
an electronic memory removably coupled to said housing, said electronic memory being in electrical communication with said camera such that electronic memory stores video recorded by said camera; and
a power supply coupled to said housing, said power supply comprising a cord extending away from said housing, said cord being electrically coupled to said camera, said cord having a distal end with respect to said housing, said distal end having a plug electrically coupled thereto, said plug being configured to be electrically coupled to a power source.
